(19) United States
(12) Patent Application Publication
(10) Pub. No.: US 2019/0145900 A1
(43) Pub. Date: May 16, 2019

(54) System for Implementing Paper Diagnostic Testing

(71) Applicant: Sanguine Diagnostics, Plymouth, MI (US)

(72) Inventor: Dominic Gelfuso, Plymouth, MI (US)

(21) Appl. No.: 16/304,451

(22) PCT Filed: May 25, 2017
(86) PCT No.: PCT/US2017/034561

§ 371 Date: Nov. 26, 2018

Related U.S. Application Data
(60) Provisional application No. 62/341,293, filed on May 25, 2016.
(60) Provisional application No. 62/487,091, filed on Apr. 19, 2017.

(51) Int. Cl. G01N 21/78; G16H 10/40; B01L 3/00; A61B 10/00

(52) U.S. Cl. CPC G01N 21/78; G16H 10/40; B01L 3/5029; A61B 10/0045; B01L 2300/18; B01L 2400/0406; B01L 2300/0681; B01L 2300/0816; B01L 2300/0867; A61B 2010/0006

(57) ABSTRACT

A paper diagnostic test implementation system is disclosed. The system can include: a test sample access port; a housing portion configured to hold a plurality of paper diagnostic tests; and at least one channel with a first end of the channel in fluid communication with the housing portion and a second end of the channel in fluid communication with the test sample access port, such that a test sample is delivered from the test sample access port to the housing portion by capillary action. The housing portion of the system may also include a plurality of test chambers each configured to hold a paper diagnostic test. The system may also include at least one temperature controller removeably coupled to the housing portion for controlling a temperature of the plurality of test chambers and a results analyzer configured to read and store a colorimetric result of the paper diagnostic tests.

This application claims priority to U.S. Provisional Application No. 62/341,293, filed May 25, 2016; and U.S. Provisional Application No. 62/487,091, filed on Apr. 19, 2017, each of which is incorporated by reference herein.

### BACKGROUND

### Technical Field

Aspects of the disclosure relate generally to the field of paper-based diagnostics. More specifically, embodiments of the present disclosure include a system for implementing paper diagnostic testing of samples.

### Related Art

The analysis of samples, for example, biological samples is useful for diagnosis of disease/conditions and for monitoring the health of individuals and populations. Additionally, the analysis of samples, such as, agricultural samples is useful for detecting the presence of pathogens and monitoring the environment or food sources. Rapid and accurate analysis of these samples is important for early detection and treatment to prevent the spread and minimize the impact of these diseases, pathogens, etc. As a result, conventional laboratory and clinical testing has shifted focus to what is generally referred to as “point-of-care testing” (POCT) or “patient centric care.” Key market drivers of POCT systems include: low-cost for widespread availability including developing regions lacking infrastructure; rapid and accurate analysis for early detection and treatment; individualized and custom patient testing; small sample sizes for convenience and safety; portability and durability for transport to the point-of-care; ease-of-use for a wide range of user skillsets; etc.

One breakthrough of POCT includes the development of paper-based devices (PADs). PADs may be easily fabricated from inexpensive materials, for example, by printing hydrophobic barriers using a sold ink printer (e.g. wax) and utilizing the wicking or capillary properties of the paper to transport the sample to desired colorimetric assay regions in the paper. PADs are generally small in size, inexpensive to produce, require small sample size, and are easy to use based on the visible and non-visible colorimetric nature of the output. Further, test times for PADs are rapid and range anywhere on the order of seconds to minutes compared to laboratory testing which can range on the order of hours to days.

Current implementation of PADs include directly applying the test sample to the PAD. Direct sample application may: pose a bio-hazard risk to those who may come into contact with the device; expose the sample and PAD to contamination resulting in faulty results; oversaturate the PAD with excessive sample volume; and require individual samples or individual application of samples to each PAD resulting in increased sample sizes and test times. Another implementation of PADs includes fabricating multiple PADs on a single substrate and directly applying the test sample to the substrate. Fabrication of multiple PADs on a single substrate may: limit the combination and number of tests; and limit the arrangement of tests in addition to the issues associated with direct sample application listed above.

### SUMMARY

A first aspect of the disclosure provides a paper-based diagnostics implementation system, the system including: a test sample access port; a housing portion configured to hold a plurality of paper diagnostic tests; and at least one channel wherein a first end of the channel is in fluid communication with the housing portion and a second end of the channel is in fluid communication with the test sample access port, such that a test sample is delivered from the test sample access port to the housing portion by capillary action.

A second aspect of the disclosure provides a paper-based diagnostics implementation system, the system including: at least one test sample access port; a housing portion, the housing portion including a plurality of test chambers wherein each of the test chambers is configured to hold a paper diagnostic test; and a plurality of channels wherein a first end of each channel is in fluid communication with a respective test chamber of the housing portion and a second end of each channel is in fluid communication with the at least one test sample access port, such that a test sample is delivered from the at least one test sample access port to the respective test chamber by capillary action via the plurality of channels.

A third aspect of the disclosure provides a paper-based diagnostics implementation system, the system including: a test sample access port; a housing portion, the housing portion including a plurality of test chambers wherein each of the test chambers is configured to hold a paper diagnostic test; a plurality of channels wherein a first end of each channel is in fluid communication with a respective test chamber of the housing portion and a second end of each channel is in fluid communication with the test sample access port, such that a test sample is delivered from the test sample access port to each test chamber by capillary action through the respective channel; a plurality of air filter channels for releasing air from the system, each of the plurality of air filter channels in fluid communication with a respective test chamber of the plurality of test chambers; at least one temperature controller removeably coupled to the housing portion for controlling a temperature of the plurality of test chambers; and a results analyzer configured to read and store a colorimetric result of the paper diagnostic tests.

### BRIEF DESCRIPTION OF THE DRAWINGS

The embodiments of this disclosure will be described in detail, with reference to the following figures, wherein like designations denote like elements, and wherein:

FIG. 1 shows an example paper diagnostic test implementation system, according to embodiments of the disclosure.

FIG. 2 shows an example layout of a paper diagnostic test implementation system, according to embodiments of the disclosure.

FIG. 3 shows an example layout of a paper diagnostic test implementation system, according to embodiments of the disclosure.

FIG. 4 shows an example layout of a paper diagnostic test implementation system, according to embodiments of the disclosure.

FIG. 5 shows an example layout of a paper diagnostic test implementation system, according to embodiments of the disclosure.

FIG. 6 shows an example layout of a paper diagnostic test implementation system, according to embodiments of the disclosure.

FIG. 7 shows a paper diagnostic test implementation system including a temperature controller, according to embodiments of the disclosure.

FIG. 8 shows a paper diagnostic test implementation system including a results analyzer, according to embodiments of the disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings. The drawings may be rotated in any direction and are not limited to a particular orientation.

### DETAILED DESCRIPTION

In the following description, reference is made to the accompanying drawings that form a part thereof, and in which is shown by way of illustration specific embodiments in which the present teachings may be practiced. These embodiments are described in sufficient detail to enable those skilled in the art to practice the present teachings and it is to be understood that other embodiments may be used and that changes may be made without departing from the scope of the present teachings. The following description is, therefore, merely illustrative.

Embodiments of the present disclosure provide a system for implementing paper based diagnostic devices (PADs), for example micro-PADs (gADs). Among other things, the system for implementing PADs may include a housing portion for holding PADs, a sample access port for loading a test sample, and channels for delivering the test sample to the PADs. The paper test housing portion may, for example include test chambers for the PADs. The system may also include a temperature controller for regulating the temperature of the sample and PAD during testing, and a results analyzer for reading and/or transferring the results of the PAD test for analysis. Embodiments of the present disclosure can allow for a variety of desired tests to be performed on test sample(s) in a centralized location using a variety of PADs. Embodiments of the present disclosure may also, for example, protect the test sample(s), and PADs from contamination, and/or physical damage.

PADs may include any diagnostic device made of patterned paper. A μPAD may include any diagnostic device made of patterned paper which utilizes conventional microfluidic concepts. For example, traditional fluidic or microfluidic devices are generally fabricated by etching or molding channels into substrates such as glass, silicon, polymers and/or plastics. PADs are generally fabricated by patterning hydrophobic barriers into sheets of paper to form hydrophilic channels which utilize capillary forces for sample distribution. While current dimensions of PADs or μPADs may be on the order of centimeters to decimeters, the system described herein may be adapted for any changes in dimensions of PADs or μPADs, for example, as a result of improvements in PAD fabrication technology.

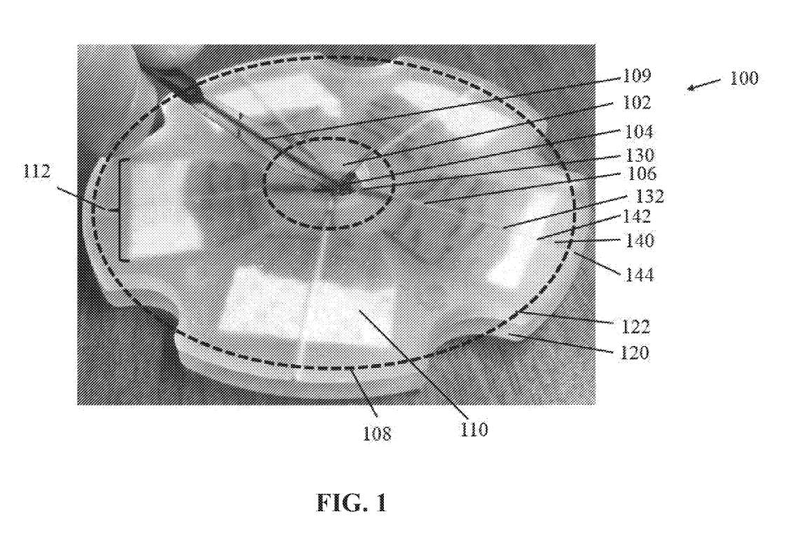

Referring to the drawings, FIG. 1 shows an example of the paper-based diagnostics implementation system 100, according to embodiments of the disclosure. System 100 may include a test sample access port 102 for introducing a test sample 104 to system 100. Test sample access port 102 may, for example, be in fluid communication with channel(s) 106 for delivering test sample 104 to a housing portion 108 of system 100. Housing portion 108 may, for example, hold a plurality of PADs 110 for testing the sample. The test sample 104 may be introduced to system 100 at test sample access port 102 and channel(s) 106 may deliver test sample 104 to housing portion 108 where it may contact and be tested by PAD(s) 110 positioned in the housing portion.

As shown in FIG. 1, system 100 may include a base portion 120 and a cover portion 122 to allow for loading and sealing PAD(s) 110 into the system. For example, PAD(s) 110 may be positioned in housing portion 108 in base portion 120 and cover portion 122 may be attached to the base portion to seal PAD(s) 110 in the housing portion. Cover portion 122 may be attached to base portion 120, for example, by fusion bonding, adhesive, and/or any other now known or later developed techniques for binding the material(s) used for system 100. Although FIG. 1 shows system 100 including two portions, cover portion 122 and base portion 120, system 100 may include any number of desirable portions for loading PADs in system 100.

Components of system 100, e.g. test sample access port 102, housing portion 108, and channel(s) 106, may be formed in base portion 120 and/or cover portion 122. For example, test sample access port 102, housing portion 108 and channel(s) 106 may be formed in base portion 120 and cover portion 122 may be attached the top of base portion 120 to seal desired portions of system 100. In another non-limiting example, a first portion of test sample access port 102, housing portion 108, and/or channels 106 may be formed in base portion 120, and a second portion of test sample access port 102, housing portion 108 and/or channel(s) 106 may be formed in cover portion 122 such that base portion 120 and cover portion 122 may be combined to form a test sample access port 102, housing portion 108 and/or channel(s) 106. System 100, including, for example, test sample access port 102, housing portion 108 and/or channel(s) 106 may be formed, for example, by micromachining, etching (e.g. laser etching), injection molding, 3-dimensional printing, and/or any other now known or later developed techniques for processing the material of system 100.

System 100 may include polymers, thermoset polyesters, poly-methylmethacrylate (PMMA), polydimethylsiloxane (PDMS), glass and/or any other now known or later developed materials desirable for holding PADs and/or test samples. System 100 may include, for example, an optically transparent materially such that the results of the PADs are visible through the system. In another example, system 100 may include a non-optically transparent material such that the PADs must be removed to view and analyze the results of the PADs. Base portion 120 and cover portion 122 may include the same material or different materials.

Test sample access port 102 may be sized and configured to receive test sample 104. For example, blood, urine, runoff water, saliva, and/or any other desirable sample for testing by PADs 108 within system 100. Test sample port 102 may, for example, be sized and configured to interface with a source 109 for the test sample. As shown in FIG. 1, source 109 may include a capillary blood collection tube. In non-limiting examples not shown, test sample access port 102 may be configured to interface with, for example, a blood bolus on a patient's finger (e.g. “fingerstick” sample), a pipette, a swab, and/or any other now known or later developed sample source. Test sample access port 102 may be formed, for example as an opening in system 100. In other non-limiting examples, test sample access port 102 may include an intravenous (IV) screw-lock port, needle puncture applicator, etc. Test sample access port 102 may be formed, for example, by micromachining, etching (e.g. laser etching), injection molding, 3-dimensional printing, and/or any other now known or later developed techniques for forming an opening in system 100. Although test sample access port 102 is shown in FIG. 1 as being located in the center of system 100, it is understood that test sample access port 102 may be formed in any desirable location on system 100 for introducing test sample 104. Test sample access port 102 may include any cross-sectional geometry desirable for receiving test sample 104. Non-limiting examples of cross-sectional geometry for channel(s) 106 include substantially circular, substantially square, substantially oval, substantially rectangular and substantially semi-circular. For example, as shown in FIG. 1, test sample access port 102 may include a substantially circular cross-sectional geometry. Additionally, although test sample access port 102 is shown as a single access port, as will be discussed herein, test sample access port 102 may include any number of access ports as may be desirable for receiving test samples to be delivered to PADs in system 100.

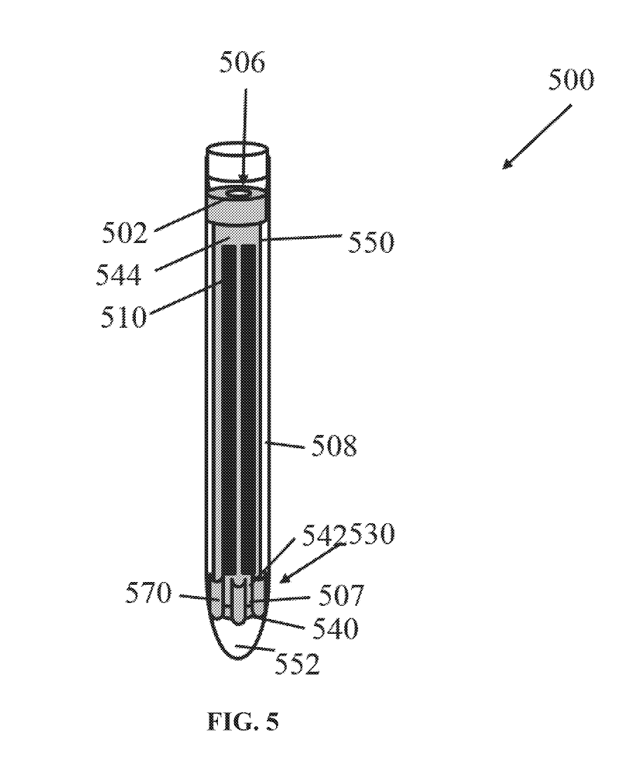

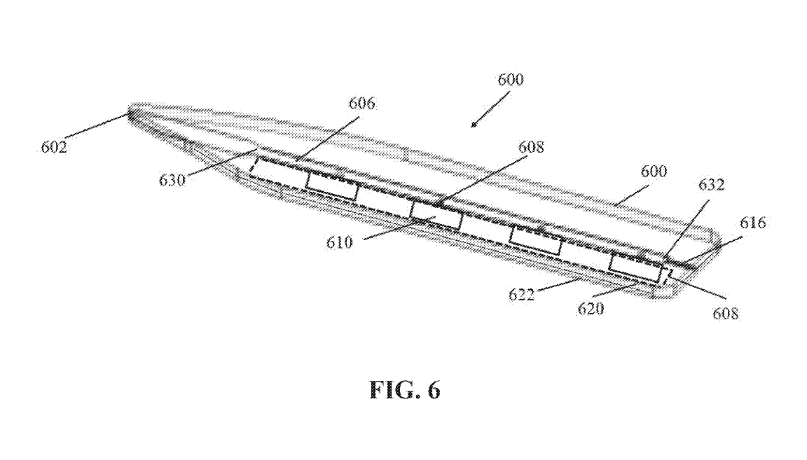

Housing portion 108, may be sized and configured to hold a plurality of PADs 110 for introduction to test sample 104 by channel(s) 106. For example, housing portion 108 may hold each PAD 110 such that the test sample 104 delivered by channels 106 is delivered to an input channel (not shown) of the PAD 110. As shown in the example of FIG. 1, housing portion 108 may include a plurality of test chambers 112, each of the test chambers sized and configured to hold an individual PAD 110. In another example, as shown in FIG. 6, housing portion 608 may hold each individual PAD 610 in place by friction, for example, by positioning the PAD between two surfaces of the housing portion. In another example, shown in FIG. 5, housing portion 508 may hold each individual PAD 510 in place by a central shaft. The method for housing portion 108 to hold PAD 110 in place is not limited to these examples.

Although FIG. 1, shows housing portion 108 and test chambers 112 or housing portion 108 as being configured to hold a specific size/dimension, number, and/or geometry of PAD(s) 110, the housing portion and test chambers 112 may be configured to hold any size, number and/or geometry of any now known or later developed PAD(s). As will be discussed herein, housing portion 108 may include any desirable layout for system 100 to hold PADs 110 for testing a test sample 104 delivered to the PAD by channel(s) 106. For example, as shown in FIG. 1, housing portion 108 may include five test chambers 112 radially distributed around a single test sample access port 102. Housing portion 108 may be formed, for example, by micromachining, etching (e.g. laser etching), injection molding, 3-dimensional printing, and/or any other now known or later developed techniques for processing the material of system 100.

Housing portion 108 of system 100 may be configured to hold specific PADs for a specific panel of tests on a specific sample. For example, panel tests may include, but are not limited to: airport screening panel including PADs for detecting Human Immunodeficiency Virus (HIV), Zika Virus, Brucella, Ebola, Severe Acute Respiratory Syndrome (SARS), etc.; sexually transmitted disease (STD) panel including PADs for detecting HIV, Syphilis, Chlamydia, Human Papillomavirus (HPV) and/or any other STDs; common clinic panel including PADs for example for detecting Pharyngitis (Strep Throat), Influenza, Staphylococcus, Acute Viral Rhinopharyngitis or Acute Coryza (Common Cold) and/or any other common ailments; and a blood donor panel including PADs for detecting Chagas Disease, Hepatitis B Virus, Hepatitis C Virus, type 1 HIV, type 2 HIV, Human T-Lymphotropic Virus (HTLV I/II), Syphilis, West Nile Virus, etc.

Channel(s) 106 may be sized and configured to deliver test sample 104 received by test sample access port 102 to housing portion 108 of system 100. As shown in the example of FIG. 1, a first end 130 of channel(s) 106 may be in fluid communication with test sample access port 102, and a second end 132 of channel(s) 106 may be in fluid communication with housing portion 108 which may, for example, include test chambers 112. Channel(s) 106 may be sized and configured to deliver test sample 104, for example, by capillary action. For example, the diameter, cross-sectional geometry, and length of each channel 106 may be selected based on the type and/or volume of test sample 104 and the number of PADs 110 in system 100. A diameter of channel(s) 106 may range, for example, from approximately 0.25 millimeters (mm) to approximately 3 mm. A Length of each channel 106 may range, for example, from approximately 5 mm to approximately 145 mm. A volume of test sample 104 required for each PAD may range, for example, from approximately 1 microliter (μL) to approximately 100 μL. A volume of test sample 104 in system 100 may, for example, be determined based on the number of PADs in system 100 and the required volume of test sample 104 for each PAD. In another example, not shown, channel(s) 106, may be sized and configured to deliver test sample 104 by producing a pressure gradient, e.g. a pressure difference driven by a mechanical device such as a syringe or plunger; a latent pressure difference created by removing the air in system 100 during manufacturing, etc.

As will be discussed herein, system 100 may include any number and/or layout of channel(s) 106 as may be desirable for delivering test sample 104 to PADs 110 in housing portion 108 of system 100. For example, as shown in FIG. 1, system 100 may include five channel(s) 106 extending radially from test sample access port 102, the second end 132 of each channel in fluid communication with a respective test chamber 112 and the first 130 end of each channel in fluid communication with a single test sample access port 102. Channel(s) 106 may include any cross-sectional geometry as may be desirable for delivering test sample 104 to housing portion 108. Non-limiting examples of cross-sectional geometry for channel(s) 106 include substantially circular, substantially square, substantially oval, substantially rectangular and substantially semi-circular. Channel(s) 106 may be formed for example, by micromachining, etching (e.g. laser etching), injection molding, 3-dimensional printing, and/or any other now known or later developed techniques for processing the material of system 100.

As shown in FIG. 1, system 100 may also include air filter channel(s) 140 in fluid communication with housing portion 108 and the surrounding environment of system 100 for releasing air (not labeled) from system 100 during the introduction, delivery, and/or processing of test sample 104 in the system. As will be discussed herein, system 100 may include any number, size, and layout of air filter channel(s) 140 as may be desirable for delivering test sample 104 to PADs 110. For example, as shown in FIG. 1, system 100 may include five air filter channel(s) 140 directed radially from test sample access port 102, a proximal end 142 of each air filter channel 140 in fluid communication with a respective test chamber 112 of housing portion 108, and a distal end 144 in fluid communication with the surrounding environment of system 100. Each air filter channel 140 may include any cross-sectional geometry desirable for filtering air out of system 100. Non-limiting examples of cross-sectional geometry for channel(s) 106 include substantially circular, substantially square, substantially oval, substantially rectangular and substantially semi-circular. As shown in the example of FIG. 1, air filter channels 140 may include a substantially circular cross-sectional geometry.

As will be discussed herein, although FIG. 1 shows system 100 having a specific layout and number of components (e.g. test sample access port 102, housing portion 108 and channel(s) 106), system 100 may include any layout, and number of components as may be desirable for testing a sample using PADs. Particular embodiments of the invention are configurable and/or operable to accommodate varying parameters and/or requirements among the plurality of PADs, such as, for example, sample volume, sample delivery rate, PAD dimensions, PAD shape, etc.

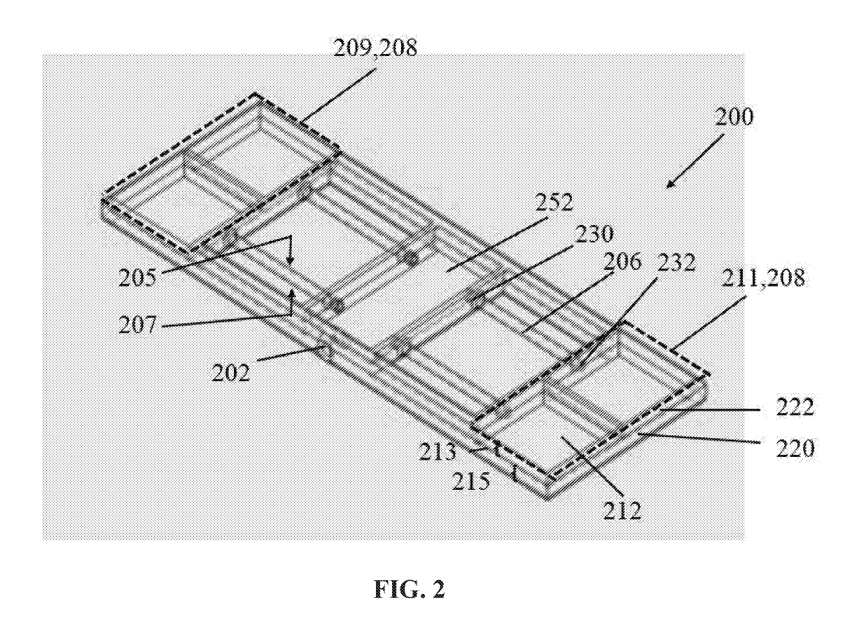

FIG. 2 shows an example layout of a paper diagnostic implementation system 200, according to embodiments of the disclosure. System 200 may include a single test sample access port 202 on a sidewall 250 of a base portion 220. Test sample access port 202 may include a substantially circular cross-sectional geometry. System 200 may include one substantially rectangular reservoir 252 formed in base portion 220. Reservoir 252 may be in fluid communication with test sample access port 202 and channel(s) 206. System 200 may include four channels 206. A bottom portion 207 of each channel 206 may be formed in base portion 220 and top portion 205 of each channel 206 may be formed in cover portion 222 such that the channel is formed and sealed when the base portion and cover portion are aligned and attached. An end 230 of each channel 106 may be in fluid communication with reservoir 252. As shown in FIG. 2, system 200 may include two of the four channels 206 opposing one another on either side of reservoir 252. A first end 232 of each channel 206 may be in fluid communication with the housing portion 208 of system 200. Channels 206 may include, for example, a substantially circular cross-sectional geometry. Housing portion 208 may include a first portion 209 and a second portion 211. First portion 209 may include two test chambers 212 and second portion 211 may include two test chambers 212. A top portion 213 of each test chamber 212 may be formed in cover portion 222 of system 200 and a bottom portion 215 of each test chamber 212 may be formed in base portion 220 of system 200 such that the chambers are formed and sealed when base the base portion and cover portion are aligned and attached. Test chambers 212 of each portion 209, 211 of housing portion 208 may be adjacent to one another. Each test chamber 212 may be in fluid communication with a respective channel 106. As shown in FIG. 2, system 200 may not include air filter channels, although this is neither necessary nor essential.

As shown in FIG. 2, system 200 may include a reservoir 252 for storing excess amounts of test sample 104. Although FIG. 2 shows a single, substantially rectangular reservoir 252 centrally located in system 200 and in fluid communication with each channel 206 and test sample access port 202, system 200 may include any number of reservoirs 252 in any location in system 200 as may be desirable for storing any excess amounts of test sample 104. In a non-limiting example, not shown, reservoir 252 may be in thermal communication with a test chamber 212. Reservoir 252 may include any shape and or size desirable for storing an excess volume of test sample 104 in system 200. Reservoir 252 may be formed, for example, by micromachining, etching (e.g. laser etching), injection molding, 3-dimensional printing, and/or any other now known or later developed techniques for processing the material of system 200.

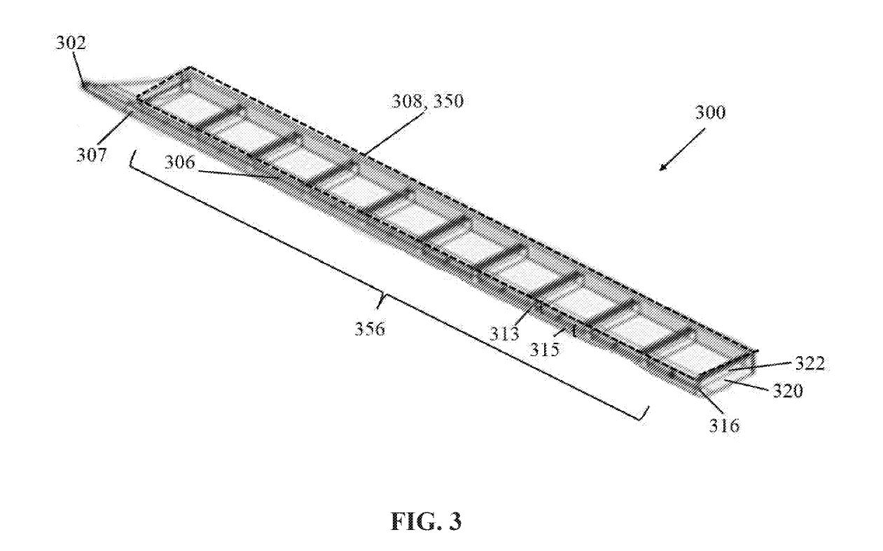

FIG. 3 shows a paper diagnostic implementation system 300, according to embodiments of the disclosure. System 300 may include a housing portion 308 including a linear array 350 of ten test chambers 312. As shown in FIG. 3, a bottom portion 315 of the test chambers 312 may be formed in a base portion 320 of the system 300. A top portion 313 of the test chambers 312 may be formed in a cover portion 322 of the system 300 such that test chamber 312 is formed and sealed when the base portion and cover portion are aligned and attached. System 300 may also include a main channel 307 formed in base portion 320. First channel 307 may extend along the side of each of the ten chambers 312 of the linear array. A first end 340 of main channel 307 may be in fluid communication with a test sample access port 302. System 300 may also include a linear array 356 of ten laterally separated channels 306 formed in base portion 320. Linear array 356 of channels 306 may be disposed between main channel 307 and linear array 350 of test chambers 312. A first end (not labeled) of each channel 306 of linear array 356 may be in fluid communication with a respective test chamber 312 of linear array 350. A second end (not labeled) of each channel 306 may be in fluid communication with main channel 307. As shown in FIG. 3, system 300 may include an air filter channel 316 formed in base portion 220 of the system. A second end 346 of main channel 307 may include an air filter opening 316. Main channel 307, air filter opening 316, test sample access port 302, and channels 306 may include, for example, a substantially circular cross-sectional geometry. As shown in FIG. 3, system 300 may not include a reservoir, although this is neither necessary nor essential.

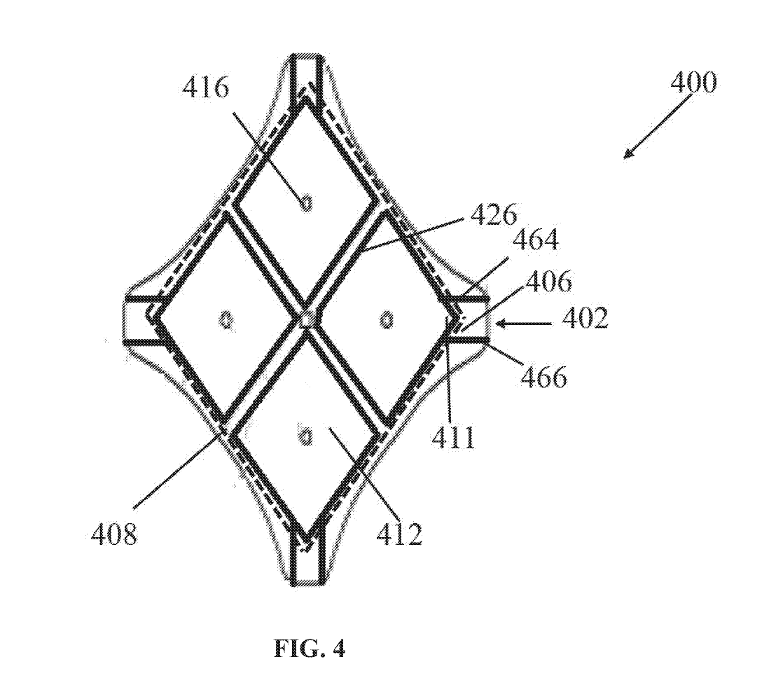

FIG. 4 shows a top view of a paper diagnostic implementation system 400, according to embodiments of the disclosure. System 400 may include a housing portion 408 including four test chambers 412 in disposed in a diamond configuration such that a side 426 of each test chamber 412 is adjacent to the side of an adjacent test chamber 412. Each test chamber 412 may, for example, be formed in a base portion (not shown). System 400 may also include four channels 406 and four test sample access ports 402. Each channel 406 may extend outward from a corner 411 of a respective test chamber 412. Channels 406 and access ports 402 may be formed, for example, in a bottom portion (not shown) of system 400. A first end 464 of each channel 406 may be in fluid communication with corner 411 of a respective test chamber 412. A second end 466 of each channel 406 may be in fluid communication with a respective test sample access port 402. System 400 may also include four air filter channels 416, each air filter channel formed in cover portion (not shown) positioned at the center of a respective test chamber 412. Each air filter channel 416 may be in fluid communication with a respect test chamber 412 and the surrounding environment of system 400. Channels 406 and access ports 402 may include, for example, a substantially rectangular cross-sectional geometry. Air filter channels 416 may include, for example, a substantially rectangular cross-sectional geometry. System 400 may not include a reservoir, although this is neither necessary nor essential.

FIG. 5 shows a paper diagnostic implementation system 500, according to embodiments of the disclosure. System 500 may include a channel 506 extending through a shaft 550. Shaft 550 may be positioned within a housing portion 508 of system 500. A first end (not labeled) of channel 506 may be in fluid communication with a test sample access port 502. A second end (not labeled) of channel 506 may be in fluid communication with a reservoir 552. As shown in FIG. 5, shaft 550 may include feet 570 at a bottom end 530 of shaft 550 radially distributed around the shaft. Channels 507 may be formed in the spaces between feet 570. A first end 540 of each channel 507 may be in fluid communication with reservoir 552 and a second end 542 may be in fluid communication with the housing portion 508. Housing portion 508 may include and surround outside surface 544 of shaft 550. Outside surface 544 of shaft 500 may be configured to hold PADs 510 such that each PAD is in fluid communication with a respective channel 507. Housing portion 508 may include a tube-like structure with a substantially circular cross-sectional geometry. Channel 506 and test sample access port may include a cross-sectional geometry. As shown in FIG. 5, system 500 may not include a reservoir or an air filter channel, although this is neither necessary nor essential.

FIG. 6 shows a paper diagnostic implementation system 600, according to embodiments of the disclosure. System 600 may include one test sample access port 602 at a first end 630 of a channel 606. A bottom portion (not labeled) of channel 606 and test sample access port 602, respectively, may be formed, for example, in a base portion 620 of system 600. Channel 606 and test sample access port 602 may be formed, for example, in a cover portion 622 of system 600 such that channel 606 and test sample access port 602 are sealed when the base portion and cover portion of system 600 are aligned and attached. System 600 may also include an air filter channel 616 in fluid communication with a second end 632 of channel 606. As shown in FIG. 6, channel 606 may also include a linear array of laterally separated openings 607. Each opening 607 may be in fluid communication with a housing portion 608 of system 600. Housing portion 608 may include a bottom surface (not labeled) of cover portion 622 and a top surface (not labeled) of base portion 620 of system 600 for holding PAD 110 in place between the surfaces when they are placed together. As shown in FIG. 6, channel 606, openings 607, and air filter channel 616 may include a substantially circular cross-sectional geometry. Test sample access port 602 may include a substantially rectangular cross-sectional geometry. System 600 of system 100 may not include a reservoir, although this is neither necessary nor essential.

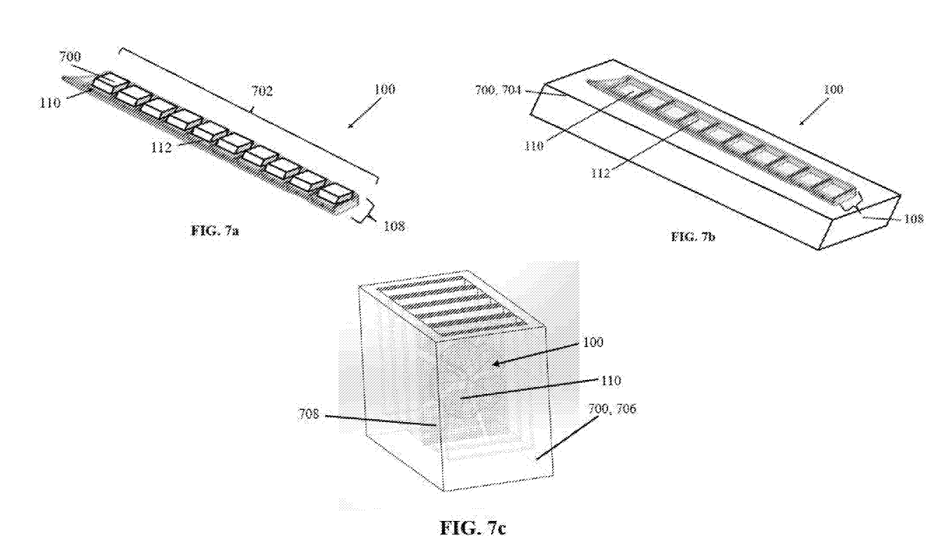

Some PADs 110 and/or test samples 104 may require specific temperatures for processing. FIGS. 7a and 7b show examples of a paper diagnostic implementation system 100 including a heat controller 700, according to embodiments of the disclosure. Heat controller 700 may, for example, control the temperature of the test sample and PAD 110 within housing portion 108 during testing of the sample. Heat controller 700 may be removably attached to system 100 as a whole, housing portion 108 of system 100, individual test chambers 112, and/or any portion of system 100 as may be desirable to control the temperature of test sample 104 and PAD 110 in housing portion 108. Heat controller 700 may include a piezoelectric temperature controller, heat pipe, vapor chamber, incubator, flexible positive temperature coefficient (PTC) heating elements, thin film heating elements, thermal coils, conductive rubber heating elements, printed heating elements, proportional-integral-derivative (PID) controller, and/or any other now known or later developed device for controlling temperature.

Heat controller 700 may include, for example, a plurality of heat controllers 702. As shown in the example of FIG. 7a, heat controller 700 may include ten heat controllers. Each heat controller of the plurality of heat controllers 702 may be in thermal communication with any portion of system 100 as may be desirable for controlling the temperature of test sample 104 and PADs 110. For example, as shown in FIG. 7a, each controller 700 may be in thermal communication with a respective test chamber 112 of system 100. Each heat controller 700 may, for example, maintain the temperature of each test chamber 112 at the same temperature and/or different temperatures. Although FIG. 7 shows ten heat controllers 700, any number of heat controllers as may be desirable for system 100 may be included.

As shown in the example of FIG. 7b heat controller 700 may include, for example, a single heat controller 704. Heat controller 704 may, for example, control a single temperature of system 100 as a whole. Heat controller 704 may control any portion of system 100 at any temperature as may be desirable for the testing test sample 104 by any type of PAD 110.

As shown in the example of FIG. 7c, heat controller 700 may include, for example, an incubator 706. Incubator 706 may be configured to hold a plurality of systems 100. Incubator 706 may be configured, for example, to be in thermal communication with the plurality of systems 100 and/or PADs 110 in system(s) 100 and/or test sample (not shown) in system(s) 100. Incubator 706 may, for example, control the temperature of each system 100 using heating elements 708. Heating elements 708 may include, for example, heat coils. Although one heat coil 708 is shown, any desirable number of heat coils may be included. Although incubator 706 is shown to hold 5 of system 100, incubator 706 may hold any desirable number of systems 100.

Although not shown, system 100 may also include, for example, alignment features (e.g. tabs, slits, etc.) for use with temperature controller 700. For example, alignment features (not shown) may indicate where system 100 should be placed with respect to temperature controller 700 for controlling the temperature of PADs 110 and/or test sample 104. Although not shown, system 100 may also include, for example, a temperature requirement indicator for use with temperature controller 700. For example, the temperature requirement indicator may, for example, be read by heat controller 700 and indicate to heat controller 700 the temperature requirements for PAD(s) 110 in system 100.

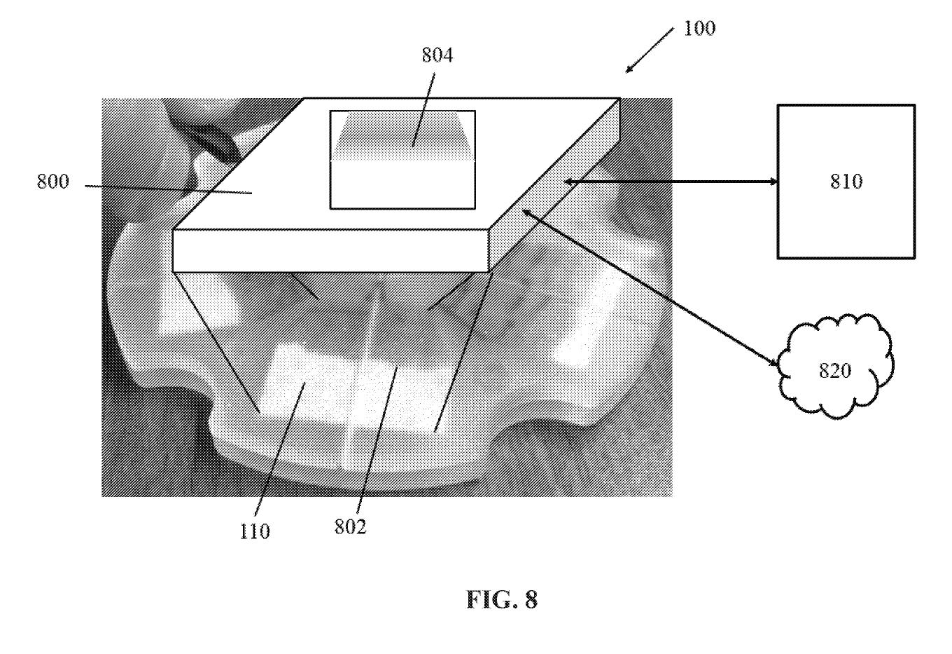

FIG. 8 shows an example of a paper diagnostic implementation system 100 including results analyzer 800, according to embodiments of the disclosure. The test results of PADs 110 in system 100 may, for example, be analyzed visually by the user. In another non-limiting example, system 100 may include a results analyzer configured to read the results of each PAD 110. For example, each PAD 110 may include an optical response 802, for example, a colorimetric response, after processing a test sample. Optical response 802 may also include, for example, a measurable artifact left as a result of processing a test sample. Optical response 802 may be visible or non-visible. For example, optical response 802 may include a response on the ultraviolet (UV) spectrum, infrared (IR) spectrum, etc. Results analyzer 800 may, for example, be configured to capture an image 804 of the optical response 802 for analysis by a processor 810. Results analyzer 800 may include, for example, a camera and or any other now known/or later developed tool for capturing an image of the test result.

Results analyzer 800 may also be configured, for example, to transfer image 804 to processor 810 for analysis of the test result and data storage. Results analyzer 800 may transfer data such as image 804 to processor 810, for example, via a network, such as the Internet, a local area network, a wide area network, and/or wireless network. Processor 810 may be a processor of a general purpose computer, special purpose computer or other programmable data processing apparatus. Processor 810 may be configured to analyze the image, e.g. by comparing color values of pixels in portions of image 804 with color values of pixels in a standard image with predetermined values associated with specific analyzed results such as the presence or lack of a particular pathogen. Processor 810 may also be configured to store image 804 and analysis results determined by the processor relating to the image. Processor 810 may, for example, be a part of system 100 and/or separate from system 100. Results analyzer 800 also may, for example, be configured to obtain the analysis results from processor 810 for display to the user of system 100. For example, processor 810 and results analyzer 800 may include a camera and processor of a handheld device such as a smart phone or tablet. Results analyzer 800 may be configured to display the analysis results from processor 810, and/or transfer the results to a tool for displaying the analysis results.

Results analyzer 800 may also be configured to determine and record a geographical location of system 100. For example, results analyzer 800 may determine the geographical location of system 100 and record the location as information associated with image 804 and/or analysis results of the image. Results analyzer 800 may be operable to access a cloud network 820. For example, results analyzer 800 may be operable to transfer and receive image 804, analysis results and/or geographical location of system 100 to and from cloud network 820 for storage. Results analyzer 800 may access cloud network 820, for example, via a network, such as the Internet, a local area network, a wide area network, and/or a wireless network.

Although not shown, system 100 may also include, for example, alignment features (e.g. tabs, slits, marks, etc.) for use with results analyzer 800. For example, alignment features (not shown) may indicate where system 100 should be placed with respect to results analyzer 800 for reading optical response 802 of PAD(s) 110.

Although temperature controller 700 and results analyzer 800 are shown to be separate components, temperature controller 700 and results analyzer 800 may be integrated into a single device.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A paper-based diagnostics implementation system, the system comprising:
a test sample access port;
a housing portion configured to hold a plurality of paper diagnostic tests; and
at least one channel wherein a first end of the channel is in fluid communication with the housing portion and a second end of the channel is in fluid communication with the test sample access port, such that a test sample is delivered from the test sample access port to the housing portion by capillary action.

2. The system of claim 1, wherein the housing portion includes a plurality of test chambers, each of the test chambers configured to hold one of the plurality of paper diagnostic tests.

3. The system of claim 1, further comprising at least one air filter channel for releasing air, the at least one air filter channel in fluid communication with the housing portion.

4. The system of claim 1, further comprising at least one temperature controller removeably attached to the housing portion and in thermal communication with the housing portion for controlling a temperature of the test sample and the plurality of paper diagnostic tests in the housing portion.

5. The system of claim 1, further comprising a results analyzer, the results analyzer configured to read a result of the plurality of paper diagnostic tests.

6. The system of claim 5, wherein the result includes a colorimetric result.

7. The system of claim 5, wherein the results analyzer is further configured to transfer the result to a processor for data storage and execution of an analysis process.

8. The system of claim 7, wherein the processor is operable to access a cloud network.

9. The system of claim 5, wherein the results analyzer is configured to obtain an analysis of the result from the processor.

10. The system of claim 5, wherein the results analyzer includes a camera.

11. The system of claim 5, wherein the results analyzer is configured to record a geographic location of the system.

12. The system of claim 1, wherein a cross-sectional geometry of the at least one channel is selected from the group consisting of: substantially square, substantially circular, substantially semicircular, substantially rectangular, and substantially oval.

13. The system of claim 1, wherein the at least one channel is operable to receive a volume of the test sample of at least 20 microliters.

14. A paper-based diagnostics implementation system, the system comprising:
at least one test sample access port;
a housing portion, the housing portion including a plurality of test chambers wherein each of the test chambers is configured to hold a paper diagnostic test; and
a plurality of channels wherein a first end of each channel is in fluid communication with a respective test chamber of the housing portion and a second end of each channel is in fluid communication with the at least one test sample access port, such that a test sample is delivered from the at least one test sample access port to the respective test chamber by capillary action via the plurality of channels.

15. The system of claim 14, wherein the at least one test sample access port includes a plurality of test sample access ports, and each channel of the plurality of channels is in fluid communication with a respective test sample access port of the plurality of test sample access ports.

16. The system of claim 14, further comprising a plurality of temperature controllers, each temperature controller removeably attached and in thermal communication with one of the test chambers for controlling a temperature of the test sample and the paper diagnostic test in the respective test chamber, and wherein the plurality of temperature controllers is configured to maintain a first temperature in a first test chamber and a second, distinct temperature in a second test chamber.

17. The system of claim 14, further comprising a results analyzer, the results analyzer configured to:
read a colorimetric result of the at least one paper diagnostic test;
record a geographical location of the system;
transfer the colorimetric result and the geographical location to a processor for an analysis process; and
obtain an analysis result of the colorimetric result from the processor.

18. The system of claim 14, wherein the plurality of test chambers are radially distributed around the access port.

19. The system of claim 14, wherein the plurality of test chambers are positioned linearly adjacent to one another.

20. A paper-based diagnostics implementation system, the system comprising:
a test sample access port;
a housing portion, the housing portion including a plurality of test chambers wherein each of the test chambers is configured to hold a paper diagnostic test;
a plurality of channels wherein a first end of each channel is in fluid communication with a respective test chamber of the housing portion and a second end of each channel is in fluid communication with the test sample access port, such that a test sample is delivered from the test sample access port to each test chamber by capillary action through the respective channel;
a plurality of air filter channels for releasing air from the system, each of the plurality of air filter channels in fluid communication with a respective test chamber of the plurality of test chambers;
at least one temperature controller removeably coupled to the housing portion for controlling a temperature of the plurality of test chambers; and
a results analyzer configured to read and store a colorimetric result of the paper diagnostic tests.
